(19) United States
(12) Patent Application Publication
(10) Pub. No.: US 2025/0252704 A1
(43) Pub. Date: Aug. 7, 2025

(54) INFORMATION PROCESSING APPARATUS, RADIATION IMAGING SYSTEM, INFORMATION PROCESSING METHOD, AND COMPUTER-READABLE STORAGE MEDIUM

(71) Applicant: CANON KABUSHIKI KAISHA, Tokyo (JP)

(72) Inventor: Hideaki MIYAMOTO, Tokyo (JP)

(21) Appl. No.: 19/191,110

(22) Filed: Apr. 28, 2025

Related U.S. Application Data
(63) Continuation of application No. PCT/JP2023/039214, filed on Oct. 31, 2023.

(30) Foreign Application Priority Data
Nov. 4, 2022 (JP) 2022-176858

(51) Int. Cl. G06V 10/25; G06V 10/12; G06V 20/70; G06V 40/10; G16H 30/40

(52) U.S. Cl. CPC G06V 10/25; G06V 10/12; G06V 20/70; G06V 40/10; G16H 30/40; G06V 2201/03

(57) ABSTRACT

An information processing apparatus is provided that comprises: a first recognizing unit configured to recognize a region of interest using an optical image including an object, which is obtained in an imaging scene of a radiation image, a second recognizing unit configured to recognize candidate information indicating a candidate of the object which is an imaging-target of the radiation image using the optical image; and a determining unit configured to determine object information indicating the object which is the imaging-target using at least one of recognition results by the first recognizing unit and the second recognizing unit.

### CROSS-REFERENCE TO RELATED APPLICATIONS

This application is a Continuation of International Patent Application No. PCT/JP2023/039214, filed Oct. 31, 2023, which claims the benefit of Japanese Patent Application No. 2022-176858, filed Nov. 4, 2022, both of which are hereby incorporated by reference herein in their entirety.

### BACKGROUND OF THE INVENTION

### Field of the Invention

This disclosure relates to an information processing apparatus, a radiation imaging system, an information processing method, and a computer-readable storage medium.

In recent years, in radiation imaging for a medical examination, imaging assist by optical image has come to be performed, in which an optical image is obtained by performing optical imaging of an imaging scene and an additional information obtained by analyzing the optical image is provided to an operator together with the live image. Japanese Patent Application Laid-Open No. 2020-199163 provides a mechanism for performing efficient radiation imaging without depending on the skill and experience of a radiographer by determining an imaging position of an object from an optical image and outputting information on the suitability of the imaging position.

In the technique described in Japanese Patent Application Laid-Open No. 2020-199163, the imaging position of the object is determined from the optical image. However, in the image analysis technique applied to such imaging position determination, there is a problem of further continuous improvement in accuracy in a wider range of objects and in imaging-condition.

For example, in the radiation imaging, in a case where an optical image is obtained by an optical imaging means which can capture the entire scene of the radiation imaging in the field of view, in addition to the object, a person other than the object, such as an operator of a radiation imaging apparatus or helper, may appear in the optical image. In this case, it is necessary to narrow down which one is the object from a plurality of object site candidates that are captured in the image before determining the imaging position of the object.

Therefore, in an embodiment of the present disclosure, one of the objectives is to obtain information for narrowing down an object, which is an imaging-target of radiation imaging, from a plurality of object candidates in the optical image.

### SUMMARY OF THE INVENTION

An information processing apparatus according an embodiment of the present disclosure includes: a first recognizing unit configured to recognize a region of interest using an optical image including an object, which is obtained in an imaging scene of a radiation image, a second recognizing unit configured to recognize candidate information indicating a candidate of the object which is an imaging-target of the radiation image using the optical image; and a determining unit configured to determine object information indicating the object which is the imaging-target using at least one of recognition results by the first recognizing unit and the second recognizing unit.

Further features of the present invention will become apparent from the following description of exemplary embodiments with reference to the attached drawings.

### BRIEF DESCRIPTION OF THE DRAWINGS

FIG. 1A is a diagram for illustrating a schematic configuration of a radiation imaging system according to an embodiment.

FIG. 1B is a diagram for illustrating a schematic configuration of an information processing apparatus according to the embodiment.

FIG. 2 is a flowchart for showing a processing procedure according to a first example.

FIG. 3 is a diagram for explaining an example of an optical image according to the first example.

FIG. 4 is a diagram for illustrating an example of an output of the first recognizing unit according to the first example.

FIG. 5 is a diagram for illustrating an example of an output of the second recognizing unit according to the first example.

FIG. 6 is a diagram for illustrating a display example on a display unit according to the first example.

FIG. 7 is a diagram for illustrating examples of the output of the first recognizing unit and the second recognizing unit according to a modification of the first example.

FIG. 8 is a flowchart for showing a processing procedure according to a second example.

FIG. 9 is a diagram for illustrating an example of an output of a first recognizing unit according to the second example.

FIG. 10 is a diagram for explaining a template and a marker according to the second example.

FIG. 11 is a diagram for explaining an example of an output of a recognition result integrating unit according to the second example.

FIG. 12 is a diagram for explaining an example of an output of an annotation unit according to the second example.

FIG. 13 is a diagram for explaining an example of an output of a first recognizing unit according to a third example.

### DESCRIPTION OF THE EMBODIMENTS

Hereinafter, an exemplary embodiment and exemplary examples for implementing the present disclosure will now be described in detail with reference to the drawings. However, the dimensions, materials, shapes, relative positions, the like of the components described in the following embodiment and examples can be freely set and may be modified according to the configuration of the apparatus to which the present invention is applied or various conditions. Also, in the drawings, the same reference numerals are used between the drawings to indicate identical or functionally similar elements.

In the following, the term “radiation” may include, for example, electromagnetic radiation such as X-rays and y-rays, and particle radiation such as a-rays, B-rays, particle rays, proton rays, heavy ion rays, and meson rays.

The term “machine learning model” refers to a learning model based on a machine learning algorithm. Specific algorithms of the machine learning include the nearest neighbor method, the naive Bayes method, the decision tree, the support vector machine, and the like. The deep learning, which generates characteristic amount and combining weighting factors for learning by itself using a neural networks, is also mentioned. As an algorithm using the decision tree, a method using gradient boosting such as LightGBM or XGBoost is also mentioned. It can be applied to the following embodiments and example as appropriate using available algorithms among the above. The term “teacher data” refers to training data and consists of a pair of input data and output data. The output data of training data is also called ground truth.

Furthermore, the term “learned model” refers to a machine learning model that has been performed training (learning) on a machine learning model, which accords to any machine learning algorithm such as the deep learning, using an appropriate training data (training data) in advance. The learned model has been obtained using an appropriate training data in advance, however it does not mean that further learning is not performed, and the incremental learning may be performed on the learned model. The incremental learning can be performed even after the apparatus is installed at the place of use.

As described above, in the prior art, for example, in a case where an optical image is obtained by an optical imaging means that captures the entire site of radiation imaging in the field of view, in addition to an object, a person other than the object, such as an operator of a radiation imaging apparatus or a helper, may appear in the optical image. Moreover, even in a case of imaging in which an optical imaging apparatus is attached to a radiation generator of a radiation imaging apparatus to capture a narrow range in the field of view, for example, in the case of imaging an extremity, since an imaged site is held by a hand to fix a posture, the hand that is not an imaging-target site may appear in the optical image. In such a case, it is necessary to narrow down which site is the object from among the plurality of object site candidates that have appeared in the image before determining the imaging position of the object.

On the other hand, in a radiation imaging system according to an embodiment of the present disclosure, image analysis of an optical image is performed to obtain information for narrowing down an object, which is an imaging-target of radiation imaging, from among a plurality of object candidates in the optical image. Specifically, a region of interest and object candidate information are recognized from the optical image, and the recognized region of interest and object candidate information are used to narrow down the object, which is the imaging-target of the radiation imaging, from among the plurality of object candidates in the optical image.

### (Schematic Configuration)

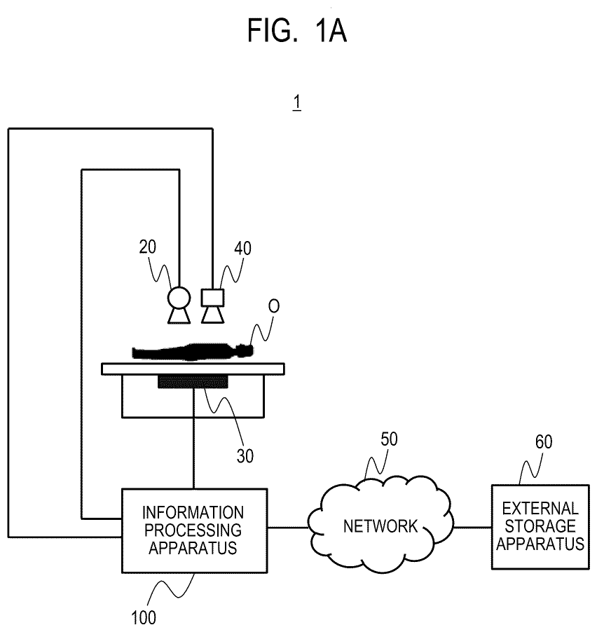

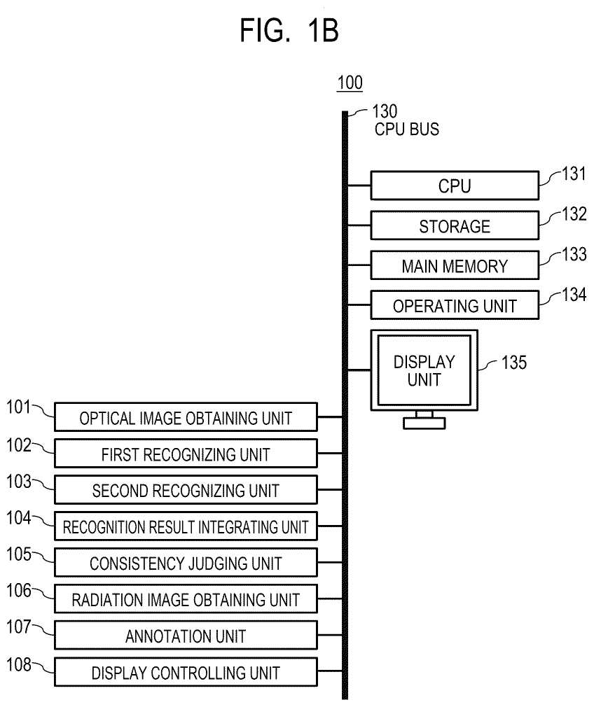

First, with reference to FIG. 1A and FIG. 1B, a radiation imaging system, an information processing apparatus, and an information processing method according to an embodiment of the present disclosure will be described. The embodiment of the present disclosure is applied to, for example, a radiation imaging system 1 and an information processing apparatus 100 shown in FIG. 1A and FIG. 1B. FIG. 1A shows the schematic configuration of the radiation imaging system 1 according to the embodiment of the present disclosure, and FIG. 1B shows the schematic configuration of an information processing apparatus 100 according to the embodiment of the present disclosure. FIG. 1A shows a state in which an object O is in a recumbent position, however the object O may be in a standing position or a sitting position, for example. An imaging table used to support the object O may be a table corresponding to the position of the object O.

The radiation imaging system 1 is provided with the information processing apparatus 100, a radiation generating apparatus 20, a radiation detector 30, and a camera 40. The information processing apparatus 100 is connected to the radiation generating apparatus 20, the radiation detector 30, and the camera 40, and can control them. The information processing apparatus 100 can perform image processing and analysis processing of various images obtained by using the radiation detector 30 and the camera 40. The information processing apparatus 100 is connected to an external storage apparatus 60 such as a server via any network 50 such as the Internet or an intranet, and can exchange data with the external storage apparatus 60. The external storage apparatus 60 may be directly connected to the information processing apparatus 100.

The radiation generating apparatus 20 includes, for example, a radiation generator such as a radiation tube, a collimator, a collimator lamp, etc., and can irradiate a radiation beam under the control by the information processing apparatus 100. The radiation beam irradiated from the radiation generating apparatus 20 transmits the object O with attenuation, and enters the radiation detector 30.

The radiation detector 30 can detect the incident radiation beam and transmit a signal corresponding to the detected radiation beam to the information processing apparatus 100. The radiation detector 30 may be any radiation detector that detects the radiation and outputs a corresponding signal, and may be configured using, for example, an FPD (Flat Panel Detector) or the like. The radiation detector 30 may be an indirect conversion type detector that converts the radiation into visible light once using a scintillator or the like, and converts the visible light into an electric signal using an optical sensor or the like, or a direct conversion type detector that directly converts the incident radiation into an electric signal.

The camera 40 is an example of an optical apparatus that performs optical imaging on the object O under the control by the information processing apparatus 100 and obtain an optical image. The camera 40 transmits the optical image obtained by the imaging to the information processing apparatus 100. The camera 40 may have any known configuration, and may be configured as a camera capable of imaging a moving image such as a video camera, or may be configured as a camera capable of imaging only a still image. The camera 40 may be configured to perform imaging with visible light, or may be configured to perform imaging with an invisible light other than radiation such as infrared light.

The information processing apparatus 100 is provided with an optical image obtaining unit 101, a first recognizing unit 102, a second recognizing unit 103, a recognition result integrating unit 104, a consistency judging unit 105, a radiation image obtaining unit 106, an annotation unit 107, and a display controlling unit 108. The information processing apparatus 100 is provided with a CPU 131, a storage 132, a main memory 133, an operation unit 134, and a display unit 135. Each unit of the information processing apparatus 100 is connected via a CPU bus 130, and can exchange data with each other.

The optical image obtaining unit 101 can control the camera 40 and obtain the optical image of the object O imaged by the camera 40. Furthermore, the optical image obtaining unit 101 may obtain the optical image of the object O from the external storage apparatus 60 or an optical apparatus (not shown) connected to information processing apparatus 100 via any network. The optical image obtaining unit 101 may obtain the optical image stored in the storage 132.

The first recognizing unit 102 performs image analysis of the optical image and can specify and recognize a region of interest in the optical image. The second recognizing unit 103 performs image analysis of the optical image and can specify and recognize object candidate information indicating a candidate of the object O, which is an imaging-target of the radiation image, in the optical image. The recognition processing by the first recognizing unit 102 and the second recognizing unit 103 will be described later.

The recognition result integrating unit 104 can determine object information indicating the object O, which is the imaging-target of the radiation image, by using at least one of the recognition results by the first recognizing unit 102 and the second recognizing unit 103. The recognition result integrating unit 104 can integrate the recognition results by the first recognizing unit 102 and the second recognizing unit 103, and determine the object information including information indicating the location, the kind, and the posture of the human body site using the integrated recognition results.

The consistency judging unit 105 can judge whether or not the object information determined by the recognition result integrating unit 104 and information about the object O included in an imaging order of the radiation image are consistent. By providing the judgement result to the operator, information processing apparatus 100 can support the judgement whether or not object information determined using optical image matches instruction of radiation imaging.

The radiation image obtaining unit 106 can control the radiation generating apparatus 20 and the radiation detector 30, perform the radiation imaging of the object O, and obtain the radiation image of the object O from the radiation detector 30. The radiation image obtaining unit 106 may also obtain the radiation image of the object O from the external storage apparatus 60 or a radiation detector (not shown) connected to the information processing apparatus 100 via any network. The radiation image obtaining unit 106 may also obtain the radiation image stored in the storage 132.

The annotation unit 107 annotates the object information determined by the recognition result integrating unit 104 to the radiation image. Here, the “annotation” means the process of embedding the object information indicating the posture and the orientation of the object O, the right or left of the site, etc. into the radiation image. The annotation unit 107 may be configured to annotate the object information to the optical image.

The display controlling unit 108 can control the display of the display unit 135. The display controlling unit 108 can cause the display unit 135 to display, for example, patient information about the patient who is the object O, an imaging-condition, parameters set by the operator, the generated optical image and radiation image, the determined object information, segmentation information, and the like. In addition, the display controlling unit 108 can cause the display unit 135 to display any display such as a button or a slider for receiving the operation of the operator, or a GUI, according to a desired configuration.

The CPU (central processing unit) 131 is an example of a processor that controls the operation of the information processing apparatus 100. The CPU 131 uses the main memory 133 to control operations from operation unit 134 and the operation of the entire apparatus according to parameters stored in the storage 132. The processor in information processing apparatus 100 is not limited to the CPU, and may include, for example, a microprocessing unit (MPU) and a graphics processing unit (GPU). The processor may also include a digital signal processor (DSP), a data flow processor (DFP), or a neural processing unit (NPU).

The storage 132 can store various images and data processed by the information processing apparatus 100. The storage 132 can store the patient information, imaging-condition, the parameters set by the operator, and the like. The storage 132 may be configured by any storage medium such as an optical disk or a memory. The main memory 133 may be configured by a memory or the like, and can be used for temporary data storage.

The operation unit 134 includes input devices for operating the information processing apparatus 100, and includes, for example, a keyboard and a mouse. The display unit 135 includes, for example, any display, and displays various types of information such as the object information, various types of images, and the like under the control by the display controlling unit 108. The display unit 135 may be a monitor for a console for operating the radiation imaging apparatus including the radiation generating apparatus 20 and the radiation detector 30, a sub-monitor installed at a position where the operator can observe the sub-monitor while supporting the positioning of the object O, or a console monitor for the radiation irradiator. Further, the display unit 135 may be configured using a device that allows the operator to reliably check the display with a small amount of eye movement, such as a head mounted display that allows the operator to work while wearing it. The display unit 135 may be configured by a touch panel type display, and in this case, the display unit 135 may also be used as the operation unit 134.

The information processing apparatus 100 may be configured by a computer provided with a processor and memory. The information processing apparatus 100 may be configured by a general computer or a computer dedicated to the radiation imaging system. The information processing apparatus 100 may be, for example, a personal computer (PC), or a desktop PC, a notebook PC, or a tablet PC (portable information terminal) may be used. Furthermore, the information processing apparatus 100 may be configured as a cloud type computer in which some components are arranged in an external device.

The optical image obtaining unit 101, the first recognizing unit 102, the second recognizing unit 103, the recognition result integrating unit 104, the consistency judging unit 105, the radiation image obtaining unit 106, the annotation unit 107, and the display controlling unit 108 may be configured by software modules executed by the CPU 131. Furthermore, each of these components may be configured by a circuit performing a specific function, such as an ASIC, or an independent device.

Next, the operation of the information processing apparatus 100 under the control by the CPU 131 will be described. First, based on imaging order information transmitted from an information management apparatus (not shown), the information processing apparatus 100 starts imaging preparation, and the optical image obtaining unit 101 starts the optical image obtainment under the control by the CPU 131. The imaging order information is information corresponding to the unit of an inspection ordered by a doctor, and includes, for example, patient information, an imaging (scheduled) date and time, and the site, the orientation, and the posture of the imaging-target based on doctor's findings. The imaging order information includes, for example, the kind of the radiation detecting apparatus to be used (for standing, lying, portable, etc.), the posture of the patient (the imaged site, the orientation, etc.), and a radiation imaging condition (tube voltage, tube current, the presence of a grid, etc.) as information required for the radiation imaging.

The optical image obtaining unit 101 controls the camera 40 to perform the optical imaging of the object O, and obtains the optical image from the camera 40. The optical image obtained by the optical image obtaining unit 101 is sequentially transferred to the main memory 133, the first recognizing unit 102, and the second recognizing unit 103 via the CPU bus 130.

The first recognizing unit 102 determines the region of interest from the transferred optical image. The second recognizing unit 103 determines the object candidate information from the transferred optical image. The processing order of the first recognizing unit 102 and the second recognizing unit 103 is not fixed, and the processing may be performed in parallel. The region of interest and the object candidate information are transferred to the recognition result integrating unit 104 via the CPU bus 130.

The recognition result integrating unit 104 integrates the transferred region of interest and object candidate information to determine the object information. The object information is transferred to the consistency judging unit 105 via the CPU bus 130. The consistency judging unit 105 compares the imaging order information with the object information, and outputs the judgement result of consistency. The optical image, the region of interest, the object candidate information, the object information, and the judgement result of the consistency are transferred to the storage 132 and the display controlling unit 108 via the CPU bus 130. The storage 132 stores the transferred various information. The display controlling unit 108 causes the display unit 135 to display the transferred various information.

The operator checks the displayed information and performs operation instruction as necessary via the operation unit 134. For example, if the judgement result of consistency is correct, the operator performs the imaging instruction of the radiation image via the operation unit 134. This imaging instruction is transmitted to the radiation image obtaining unit 106 by the CPU 131.

Upon receiving the imaging instruction, the radiation image obtaining unit 106 controls the radiation generating apparatus 20 and the radiation detector 30 to execute the radiation imaging. In the radiation imaging, the radiation is irradiated from the radiation generating apparatus 20 toward the object O, and the radiation beam transmitted through the object O with attenuation is detected by the radiation detector 30. The radiation image obtaining unit 106 obtains a signal corresponding to the intensity of radiation beam detected by the radiation detector 30 as a radiation image. This radiation image data is sequentially transferred to the main memory 133 and the annotation unit 107 via the CPU bus 130.

The annotation unit 107 annotates the transferred radiation image with the object information stored in the storage 132. The annotated radiation image is transferred to the storage 132 and the display controlling unit 108 via the CPU bus 130. The storage 132 stores the transferred annotated radiation image. The display controlling unit 108 causes the display unit 135 to display the transferred annotated radiation image. The operator can check the displayed annotated radiation image and perform an operation instruction as necessary via the operation unit 134.

### First Example

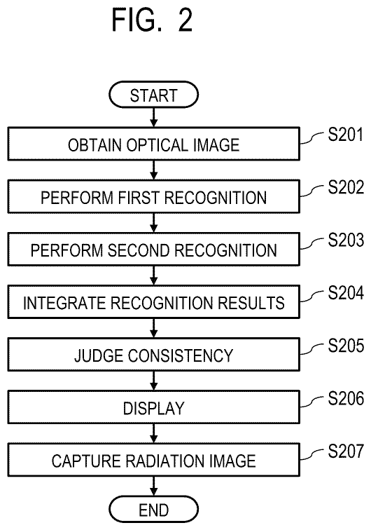

With reference to FIG. 2 to FIG. 6, a radiation imaging system, an information processing apparatus, and an information processing method according to a first example of this disclosure will be described below. In the first example, a case of confirming the consistency between the imaging order and the object information while obtaining the optical image at a predetermined frame rate using a video camera as the camera 40 and performing the radiation imaging will be described.

### (Processing Flow)

A series of processing procedures according to the first example will be described below with reference to FIG. 2. FIG. 2 is a flowchart showing the processing procedure according to the first example. When the processing procedure according to the first example starts, the processing proceeds to step S201.

### (Step S201)

In step S201, the optical image obtaining unit 101 controls the camera 40 to obtain an optical image of an imaging scene of radiation including an object of radiation imaging. In the first example, the camera 40 is a video camera attached to the radiation generator, and images the object holding an imaging posture on the radiation detector 30 arranged on the recumbent table, and outputs optical images at a predetermined frame rate.

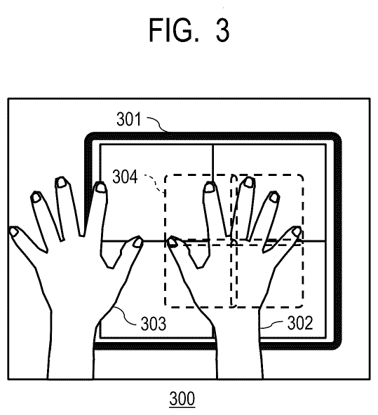

Here, an example where an imaging order is “right hand” and the radiation detector 30, a right hand 302, and a left hand 303 appears in an optical image 300 will be descried with reference to FIG. 3. FIG. 3 shows an example of the optical image according to the first example. A radiation detector region 301 in the optical image 300 is a region indicating the radiation detector 30. Further, in the example shown in FIG. 3, it is assumed that a collimator lamp is irradiated on the right hand of the object, and a collimator lamp irradiated region 304 is depicted on the right hand 302 in the optical image 300. Here, the collimator lamp is an apparatus for irradiating visible light mounted on the collimator to confirm a radiation irradiation region, which is a region where the radiation is irradiated, before the radiation irradiation. The collimator lamp irradiated region 304 generated by the collimator lamp coincides with the radiation irradiation region at the time of the radiation imaging.

### (Step S202)

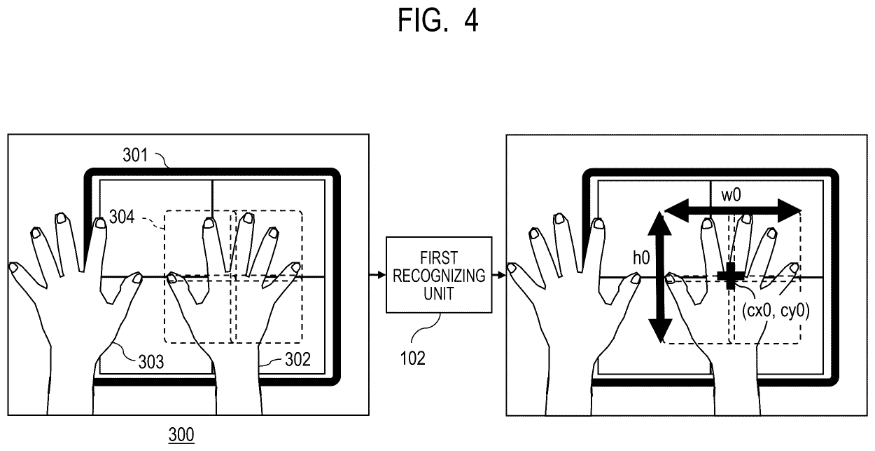

In step S202, the first recognizing unit 102 recognizes a region of interest from the optical image obtained by the optical image obtaining unit 101. The region of interest is a region where the object, which is an imaging-target to be radiation imaging, is assumed to exist. In the first example, a case where the collimator lamp irradiated region 304 is the region of interest will be described. Since the collimator lamp irradiated region 304 corresponds to the irradiation region of the radiation at the time of the radiation imaging, it corresponds to a region of interest where the object exists. Therefore, as shown in FIG. 4, the first recognizing unit 102 recognizes, for example, the circumscribed rectangle of the collimator lamp irradiated region 304 from the optical image 300, and outputs the center position (cx0, cy0), width w0, and height h0 of the circumscribed rectangle as the information indicating the recognized region of interest.

Various methods can be considered for specifying and recognizing the collimator lamp irradiated region 304. For example, a method using the fact that the collimator lamp irradiated region 304 is a region generally formed by the visible light irradiated in a dark room so as to increase the visibility in order for the operator to confirm the position of the radiation irradiation region can be used. In this case, for example, the collimator lamp irradiated region 304 can be recognized using, for the optical image, relatively simple image analysis processing such as threshold processing of a high intensity region according to the histogram analysis or rectangular region recognition processing according to the high intensity edge detection. In addition, the collimator lamp irradiated region 304 may be recognized using a cross-shaped shadow that indicates the collimator center as a feature.

Further, a neural network-based inferrer (learned model) may be generated by the machine learning to recognize the collimator lamp irradiated region 304. The inferrer in this case may be obtained by using training data that includes an optical image that captured the imaging scene of the radiation image as input data and information indicating the region of interest in the optical image as output data. The position information of the collimator lamp irradiated region may be used as the information indicating the region of interest, and the position information of the collimator lamp irradiated region may include the center position, width, and height of the collimator lamp irradiated region. As the position information of the collimator lamp irradiated region, for example, a label image in which the collimator lamp irradiated region label is labeled to the optical image may be used. In the machine learning, an inferrer more robust than the rule-based algorithm can be generated by using many training data obtained by various imaging-conditions, collimator lamp kinds, etc. In this case, the information processing apparatus 100 functions as an example of a training unit performing the training of the inferrer used for the recognition processing of the region of interest, however the first recognizing unit 102 may use inferrer trained by another training apparatus, etc.

It should be noted that the GPU can perform efficient arithmetic operation by performing parallel processing of larger amounts of data. Therefore, in a case where the training is performed a plurality of time using a machine learning algorithm such as deep learning, it is effective to perform the processing with a GPU. Therefore, in the first example, a GPU is used in addition to the CPU for processing performed by the information processing apparatus 100 which functions as an example of the training unit. Specifically, when executing a training program including a learning model, the training may be performed by the CPU and GPU cooperating to perform the arithmetic operations. Note that, with respect to the processing of the training unit, the arithmetic operations may be performed only by the CPU or the GPU. In addition, the recognition processing according to the first example may be implemented using the GPU, similarly to the training unit. If the inferrer is provided in an external apparatus, the information processing apparatus 100 may not function as the training unit.

The training unit may also include an error detecting unit and an updating unit (not shown). The error detecting unit obtains an error between output data output from the output layer of the neural network in accordance with input data input to the input layer, and the ground truth. The error detecting unit may calculate the error between the output data from the neural network and the ground truth using a loss function. Further, based on the error obtained by the error detecting unit, the updating unit updates combining weighting factors between nodes of the neural network or the like so that the error becomes small. The updating unit updates the combining weighting factors or the like by using, for example, the error back-propagation method. The error back-propagation method is a method that adjusts the combining weighting factors between the nodes of each neural network or the like so that the above error becomes small.

As the machine learning model according to the first example, for example, FCN (Fully Convolutional Network) or SegNet can be used. As the machine learning model for object recognition, for example, RCNN (Region CNN), fastRCNN, or fasterRCNN can be used. Furthermore, YOLO (You Only Look Once) or SSD (Single Shot Detector or Single Shot MultiBox Detector) may be used as the machine learning model for the object recognition in units of regions.

### (Step S203)

In Step S203, the second recognizing unit 103 recognizes the object candidate information from the optical image 300 obtained by the optical image obtaining unit 101. The object candidate information is information indicating the candidate of the object, which is an imaging-target of the radiation image, in the optical image and can include the position, the kind, and the posture of the human body site appearing in the optical image.

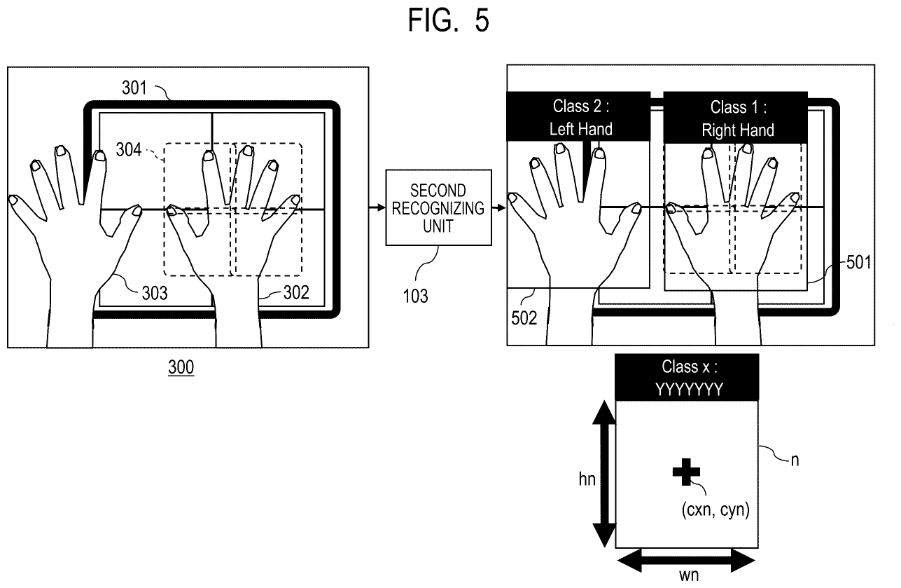

For example, taking the optical image 300 shown in FIG. 3 as an example, the second recognizing unit 103 performs the object detection of the right hand 302 and the left hand 303, and outputs their positions as different bounding boxes 501, 502 as shown in FIG. 5. Here, the center of the bounding box n is denoted as the center position (cxn, cyn), the width thereof is denoted as the width wn, and the height thereof is denoted as the height hn. The second recognizing unit 103 can output the information of the center positions, the widths, and the heights of the bounding box 501, 502 as the recognition results as the positions of the right hand 302 and the left hand 303 which are the object candidates. In the first example, the second recognizing unit 103 recognizes the position of human body site as a bounding box, but the recognition of the position of human body site is not limited thereto. For example, the second recognizing unit 103 may recognize a region along the outline of the object candidate included in the optical image as the position of the human body site, or may recognize a region of any geometric shape including the object candidate as the position of the human body site.

Further, the second recognizing unit 103 classifies the right hand 302 as the right hand and the left hand 303 as the left hand in the kinds with respect to the kinds of the right hand 302 and the left hand 303 which are the object candidate. Specifically, the second recognizing unit 103 gives the class number (label) indicating the kind to the detected object or bounding box. In the first example, the class number 1 is given to the right hand and the class number 2 is given to the left hand.

The second recognizing unit 103 may also recognize the posture of the object candidate according to the granularity of the imaging order for the consistency judgement. For example, if the object candidate is a hand, the second recognizing unit 103 can recognize the posture such as the inside and outside of the hand, the side of the hand, and the folding of the fingers with respect to the detected object or bounding box as the posture defined according to the granularity of the imaging order. In this case, the second recognizing unit 103 can give the class number corresponding to each posture to the detected object or bounding box.

There are various methods for recognition processing of the object candidate information by the second recognizing unit 103. The recognition processing of the object candidate information can be performed, for example, by using an inferrer generated by the machine learning, which is capable of performing the object detection and the class classification of parts such as the human face, the limbs, and the trunk. In particular, an inferrer for skeletal structure estimation techniques, which performed the machine learning to recognize the position and the kind of the joint site, is known to be able to perform the estimation with good accuracy generally. Further, by using clustering algorithm such as the k-means clustering, for example, with the obtained joint location and kind as characteristic amount, flexible recognition processing for the human part can be realized.

In addition, the second recognizing unit 103 may recognize the object candidate information by using an inferrer obtained by using training data including an optical image as input data and object candidate information in the optical image as output data. Here, as the object candidate information in the optical image, for example, a label image in which a label is given to a region including a site of the human body may be used. In this case, an inferrer for each target to be recognized may be generated by using training data including a label image in which a label is given according to a target to be recognized (for example, the kind or the posture of the human body site), and be used. In addition, one inferrer which infers the target to be recognized at once may be generated by using training data including a label image in which a label is given according to the combination of targets to be recognized (for example, the combination of kind and the posture of human body site), and be used. In the first example, the information processing apparatus 100 functions as an example of a training unit which performs training of the inferrer used for the recognition processing of the object candidate information, but an inferrer to which the training is performed by another training apparatus or the like may be used for the second recognizing unit 103.

### (Step S204)

In Step S204, the recognition result integrating unit 104 determines and outputs the object information which indicates the object which is the imaging-target of the radiation image by integrating the region of interest output by the first recognizing unit 102 and the object candidate information output by the second recognizing unit 103. In the first example, the object information includes information which indicates what object is to be radiation-imaged from now on.

The object information can be used to perform the consistency judgment with the imaging order. Specifically, recognition result integrating unit 104 can narrow down an object candidate which is closest to the position of the region of interest output by the first recognizing unit 102 from among the object candidate output by the second recognizing unit 103, as the object. The recognition result integrating unit 104 can also determine the narrowed kind and posture of the object candidate as the kind and the posture of the object.

The distance Dn between the region of interest and the object candidate n can be defined by the distance between the coordinates of the respective center positions. Specifically, since the center of the region of interest is the center position (cx0, cy0) and the center of the object candidate n is the center position (cxn, cyn), the distance Dn can be calculated according to the following Equation 1.

$${D}_{n} = \sqrt{{( {cx}_{0} - {cx}_{n} )}^{2} + {( {cy}_{0} - {cy}_{n} )}^{2}} \qquad ( Equation 1 )$$

The recognition result integrating unit 104 obtains the distances Dn for all object candidate n outputted by the second recognizing unit 103, recognizes an object candidate nmin with the smallest distance Dn as the object that is the imaging-target, and outputs the object information indicating the object. Further, the recognition result integrating unit 104 can output information (class) indicating the kind or the posture of the human body included in the object candidate information indicating the object candidate nmin, as the object information.

Taking the optical image 300 shown in FIG. 3 to FIG. 5 as an example, the recognition result integrating unit 104 calculates the distance D501 between the center position (cx0, cy0) of the region of interest and the center position (cx501, cy501) of the bounding box 501. The recognition result integrating unit 104 calculates the distance D502 between the center position (cx0, cy0) of the region of interest and the center position (cx502, cy502) of the bounding box 502. Since the distance D501 is smaller between the distance D501 and the distance D502, the object candidate nmin is an object candidate indicated by the bounding box 501, and the class indicating the kind of the human body site included in the object candidate is 1. Therefore, the recognition result integrating unit 104 can output the object candidate information including the bounding box 501 and its class number 1 (right hand) as the object information.

### (Step S205)

In step S205, the consistency judging unit 105 compares the imaging order with the object information output by the recognition result integrating unit 104, judges the consistency between them, and outputs “consistent” or “inconsistent” as the judgement result. In the first example, since the imaging order is the right hand and the output of the recognition result integrating unit 104 is also the class number 1 (right hand), the consistency judging unit 105 outputs “consistent” as the judgement result. On the other hand, if the imaging order is the left hand or the result of recognition result integrating unit 104 is other than the class number 1, the recognition result integrating unit 104 outputs “inconsistent” as the judgement result.

### (Step S206)

In step S206, the display controlling unit 108 causes the display unit 135 to display the optical image obtained by the optical image obtaining unit 101, the object information output by the recognition result integrating unit 104, the imaging order, and the judgement result of the consistency. The display controlling unit 108 can also cause the display unit 135 to display the region of interest output by the first recognizing unit 102 and the object candidate information output by the second recognizing unit 103.

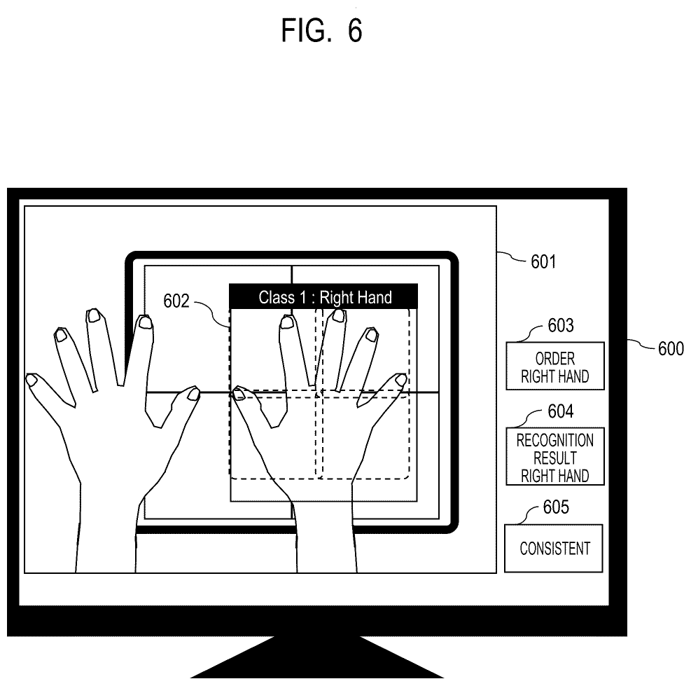

In the first example, as shown in FIG. 6, the display controlling unit 108 causes an optical image 601 to be displayed on a console monitor 600, which is an example of the display unit 135, and causes a bounding box 602 indicating the position of the right hand as object information to be displayed on the optical image 601. Furthermore, the display controlling unit 108 causes the console monitor 600 to display information 603 that information about the object included in the imaging order is the right hand, information 604 that the recognized kind of the object is the right hand, and a judgement result 605 of the consistency. FIG. 6 shows an example of the display on the display unit 135 according to the first example.

The display controlling unit 108 can also cause the display unit 135 to display the recognition result of the first recognizing unit 102 and the recognition result of the second recognizing unit 103. Further, the display controlling unit 108 can switch the respective displays on the display unit 135 according to the instruction by the operator via the operation unit 134. The operator can grasp what kind of results are output by the first recognizing unit 102, the second recognizing unit 103, the recognition result integrating unit 104, and the consistency judging unit 105 by checking these.

The information processing apparatus 100 can perform image processing such as trimming, scaling, and rotation on the optical image to be displayed based on the region of interest, the object candidate information, and the object information. Such image processing is useful, for example, when the camera 40 is positioned not at the position of the radiation generator but at a position to capture a wider field of view in the examination room. Specifically, the display controlling unit 108 can cause the display unit 135 to display an optical image limited to regions required by the operator relating to the region of interest, the object candidate information, the object information, etc. instead of the optical image which captures the wider field of view. Furthermore, the display controlling unit 108 can display an optical image with a size and rotation angle that is easy to be recognized.

In a case where the judgement result of the consistency judging unit 105 is “inconsistent”, the display controlling unit 108 may cause a warning to be displayed on the display unit 135. As the warning, for example, it may be displayed that the determined object information and information included in the imaging order are not consistent, or it may be displayed that the correction of the site and the posture of the object is urged. With such processing, it is possible to prevent the radiation imaging which is different from the radiation imaging intended by imaging order and to prevent unnecessary exposure of the object to the radiation.

### (Step S207)

In Step S207, the operator checks the optical image and the information displayed on the display unit 135, and performs an appropriate operation for performing radiation imaging via the operation unit 134. Specifically, if the result by the consistency judging unit 105 indicates that it is consistent, the operator can input an instruction to perform the radiation imaging. In addition, if the judgement result indicates that it is not consistent, or if there is some abnormality in the information displayed on the display unit 135, the operator can take necessary measures to eliminate the abnormality.

In the first example, the radiation imaging is performed according to the instruction by the operator, but the information processing apparatus 100 may determine whether or not the radiation imaging is performed. For example, the information processing apparatus 100 may start the radiation imaging if the judgement result by the consistency judging unit 105 is “consistent” for a predetermined period with respect to the optical image, which is a moving image. On the other hand, if the judgement result by the consistency judging unit 105 is “inconsistent”, the information processing apparatus 100 may not start the radiation imaging and cause the warning to be displayed on the display unit 135 by the display controlling unit 108. The warning may indicate, for example, that the recognized object information and the information included in the imaging order are not consistent, that the correction of the site and the posture of object is urged, or the like. With such processing, it is possible to prevent the radiation imaging which is different from radiation imaging intended by the imaging order, and to prevent the unnecessary exposure of the object to the radiation.

As described above, the radiation imaging system 1 according to the first example includes the radiation generating apparatus 20 and the radiation detector 30 for imaging of the object with the radiation, a camera 40 which functions as an example of an optical apparatus for capturing the optical image of the object, and the information processing apparatus 100. The information processing apparatus 100 includes the first recognizing unit 102 and the second recognizing unit 103. The first recognizing unit 102 recognizes the region of interest by using the optical image including object obtained in the imaging scene of the radiation image. The second recognizing unit 103 recognizes, by using the optical image, the object candidate information indicating the candidate of the object which is the imaging-target of the radiation image. The object candidate information includes at least one of the site, the kind, and the posture of the human body.

With the above configuration, the information processing apparatus 100 according to the first example can obtain information for narrowing down the object which is the imaging-target of the radiation imaging from among a plurality of object candidates in the optical image. Therefore, the information processing apparatus 100 can more appropriately assist the operator's judgment regarding the appropriateness of the imaging position of the object.

The information processing apparatus 100 also includes the recognition result integrating unit 104 which functions as an example of a determining unit that integrates the recognition results by the first recognizing unit 102 and the second recognizing unit 103 and determines the object information indicating the object which is the imaging-target by using the integrated recognition result. Therefore, the information processing apparatus 100 can narrow down the object which is the imaging-target of the radiation imaging from among the plurality of object candidate in the optical image by using the recognition results from the first recognizing unit 102 and the second recognizing unit 103. Therefore, the information processing apparatus 100 can more appropriately assist the operator's judgment regarding the appropriateness of the imaging position of the object. Note that the recognition result integrating unit 104 may determine the object information by using one of the distance or the overlapping area between the recognized region of interest and the object candidate indicated by the recognized object candidate information.

Furthermore, the information processing apparatus 100 further includes a consistency judging unit 105 which functions as an example of a judging unit to judge whether the object information and the information included in the imaging order of radiation image are consistent or not. The information processing apparatus 100 further includes the display controlling unit 108 that causes at least one of the optical image, the region of interest, the candidate information, the object information, and the consistency judgement result to be displayed on the display unit 135.

According to this configuration, the information processing apparatus 100 according to the first example can judge the consistency between the object information determined by the optical image analysis and the imaging order, and present the judgement result of the consistency to the operator which performs the radiation imaging. In this case, the consistency judgement function of the imaging order can be applied even in a case where a plurality of object candidates is captured on an optical image. Therefore, the information processing apparatus 100 can more efficiently assist the operator's judgement regarding the appropriateness of the imaging position of the object. The object information may include at least one of the site, the kind, and the posture of the human body.

In a case where the consistency judging unit 105 outputs a judgement result indicating inconsistency, the display controlling unit 108 may cause a warning to be displayed on the display unit 135. In this case, it is possible to prevent radiation imaging which is different from the radiation imaging intended by the imaging order and prevent unnecessary exposure of the object to radiation.

Furthermore, the first recognizing unit 102 may recognize the region of interest using output from an inferrer, which is obtained by inputting the obtained optical image into the inferrer. The inferrer in this case may be obtained by using training data including an optical image and information indicating the region of interest in the optical image. Furthermore, the second recognizing unit 103 may recognize the candidate information relating to the obtained optical image by using output from an inferrer, which is obtained by inputting the obtained optical image into the inferrer. The inferrer in this case may be obtained by using training data including an optical image and object candidate information in the optical image indicating a candidate of the object which is the imaging-target of the radiation image. With such a configuration, each of the first recognizing unit 102 and the second recognizing unit 103 can perform the recognition processing with high accuracy by using the inferrers.

Furthermore, the first recognizing unit 102 may recognize a collimator lamp irradiated region which is irradiated with a collimator lamp as the region of interest by using the optical image. According to this configuration, the information processing apparatus 100 according to the first example can obtain information on the collimator lamp irradiated region which is the region of interest as the information for narrowing down the object to one from among the plurality of object candidate. Thus, even if a plurality of object candidates appears in the optical image, the information for narrowing down the object can be efficiently obtained.

Further, the display controlling unit 108 may cause an optical image obtained by performing at least one image processing of trimming, scaling, and rotation processing on the optical image to be displayed on the display unit 135. According to such a configuration, the display controlling unit 108 can cause the display unit 135 to display an optical image, which is limited to a region required by the operator and is related to the region of interest, the object candidate information, the object information, and the like, instead of an optical image capturing a wide field of view.

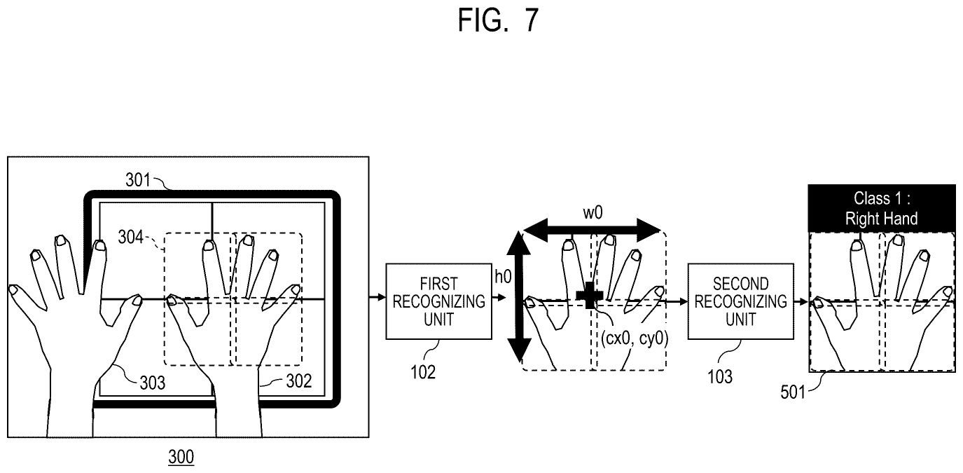

In the above description, a case where the first recognizing unit 102 in step S202 and the second recognizing unit 103 in step S203 operate independently of each other, and the results thereof are integrated by the recognition result integrating unit 104 in step S204, has been described. On the other hand, as a modified example, as shown in FIG. 7, the region of interest may be recognized by the first recognizing unit 102, and the recognition processing of the object candidate information by the second recognizing unit 103 may be applied only to the recognized region of interest. FIG. 7 shows an example of output from the first recognizing unit 102 and the second recognizing unit 103 according to this modified example.

In this case, the second recognizing unit 103 performs the recognition processing of the object candidate information in a radiation detector region 301 in the optical image indicated by the center position (cx0, cy0), the width w0, and the height h0 included in information of the region of interest output from the first recognizing unit 102. In the example shown in FIG. 7, the object candidate in the collimator lamp irradiated region 304, which is the region of interest recognized by the first recognizing unit 102, is only the right hand 302. Therefore, the second recognizing unit 103 can perform the object detection of the right hand 302 in the region of interest and output the bounding box 501 and its class number 1 as the recognition result. Then, based on the recognition result by the second recognizing unit 103, the recognition result integrating unit 104 can determine and output the object candidate information including the bounding box 501 and its class number 1 (right hand) as the object information.

In this case, the first recognizing unit 102 and the second recognizing unit 103 do not operate independently, and the result of the first recognizing unit 102 affects the performance of the second recognizing unit 103. However, it can be expected to improve the processing speed by limiting the processing target of the second recognizing unit 103. As described above, if only one object candidate is included in the region of interest, the recognition result integrating unit 104 can determine and output the object information only based on the recognition result of the second recognizing unit 103. On the other hand, a plurality of object candidates may be included in the region of interest. In this case, similarly to the first example described above, the recognition result integrating unit 104 may determine and output the object information from the object candidate information based on the distance between the recognition result by the second recognizing unit 103 and the center position of the region of interest.

In another modified example, at first, the second recognizing unit 103 recognizes a plurality of object candidate information (a plurality of candidate information) indicating a plurality of object candidates (a plurality of candidates). Thereafter, the first recognizing unit 102 may recognize the region of interest based on an area in the optical image, which is occupied by each of the plurality of object candidates indicated by the plurality of object candidate information. Specifically, the first recognizing unit 102 may recognize an object candidate, which occupies a larger area in the optical image than other object candidates among the plurality of object candidates indicated by the plurality of object candidate information, as the region of interest. In this case, the recognition result integrating unit 104 can determine the object candidate information recognized by the second recognizing unit 103 corresponding to the region of interest recognized by the first recognizing unit 102, as the object information. This configuration is based on the assumption that the object occupies the largest area in the optical image. With this configuration, the object can be accurately determined by a relatively simple method.

Further, the first recognizing unit 102 may recognize the region of interest based on the distance from each of the plurality of object candidates indicated by the plurality of object candidate information recognized by the second recognizing unit 103 to the center of the optical image. More specifically, the first recognizing unit 102 can recognize an object candidate which is closer to the center of the optical image than other object candidates among the plurality of object candidates as the region of interest. Also in this case, the recognition result integrating unit 104 can determine the object candidate information recognized by the second recognizing unit 103 corresponding to the region of interest recognized by the first recognizing unit 102, as the object information. Such a configuration has limited application conditions, such as, a condition where the camera 40 is attached to the radiation generating apparatus 20, and the like, but the object can be accurately determined by a relatively simple method.

Furthermore, the information processing apparatus 100 may recognize the presence or absence of movement of the object candidates from the optical images obtained at a predetermined frame rate, and may determine a candidate of which movement is absent as the object. Specifically, the second recognizing unit 103 recognizes a plurality of object candidate information indicating a plurality of object candidates in time series using the optical images obtained at a predetermined frame rate, and recognizes the movement of the plurality of object candidates time series. Based on the recognized movement in the time series, the first recognizing unit 102 recognizes an object candidate, whose the movement in the time series is smaller than that of other object candidates, as the region of interest. The recognition result integrating unit 104 can determine the object candidate information recognized by the second recognizing unit 103 corresponding to the region of interest recognized by the first recognizing unit 102, as the object information. This configuration is based on the assumption that the object will be stationary for the radiation imaging. Even with this configuration, the object can be accurately determined by a relatively simple method.

It should be noted that the above-described modified examples can be appropriately combined with each other. Further, if the first recognizing unit 102 recognizes the region of interest based on the recognition result by the second recognizing unit 103, the recognition result integrating unit 104 may determine and output the object information indicating the position of human body site, which is the object, based only on the recognition result by the first recognizing unit 102. In this way, according to these modified examples, the recognition result integrating unit 104 can function as an example of a determining unit that determines the object information indicating the object which is the imaging-target by using at least one of the recognition results by the first recognizing unit 102 and the second recognizing unit 103. Even in this case, the information processing apparatus 100 can narrow down the object which is the imaging-target of the radiation imaging, even in a situation where a plurality of object candidates appears in the image, and can more appropriately assist the operator's judgement regarding the appropriateness of the imaging position of the object.

Furthermore, in a case where the first recognizing unit 102 recognizes the region of interest based on the object candidate information recognized by the second recognizing unit 103, the first recognizing unit 102 may perform the function of determining and outputting the object information indicating the object based on the region of interest. In this case, the first recognizing unit 102 may determine the object information using not only the region of interest but also the object candidate information recognized by the second recognizing unit 103. Similarly, in a case where the second recognizing unit 103 recognizes the object candidate information based on the region of interest recognized by the first recognizing unit 102, the second recognizing unit 103 may perform the function of determining and outputting the object information indicating the object based on the object candidate information. In this case, the second recognizing unit 103 may determine the object information using not only the object candidate information but also the region of interest recognized by the first recognizing unit 102. Even in these cases, the information processing apparatus 100 can narrow down the object which is the imaging-target of the radiation imaging, even in a situation where a plurality of object candidates appears in the image, and can more appropriately assist the operator's judgment regarding the appropriateness of the imaging position of the object.

### Second Example

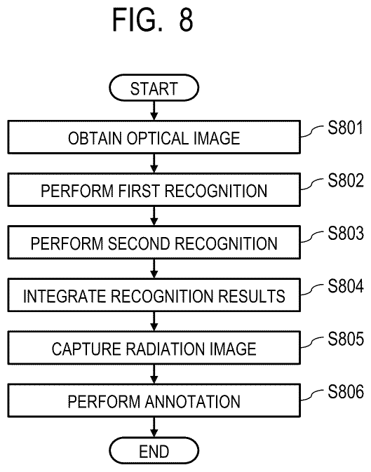

With reference to FIG. 8 to FIG. 12, a radiation imaging system, an information processing apparatus, and an information processing method according to a second example of the present disclosure will be described below. The second example describes a case in which object information is obtained while optical images are obtained at a predetermined frame rate using a video camera, and the object information is annotated to an image obtained by radiation imaging. The details described in the first example will be omitted.

Here, the annotation means to embed information indicating the posture and the orientation of an object, the left or the right of a site, etc., into a radiation image. In general, information such as the posture of a patient and the incident direction of the radiation at the time of imaging is lost in the radiation image obtained by the radiation imaging for medical examination. Therefore, by performing the annotation, information which is lost in the radiation image in which three-dimensional information has been projected to a two-dimensional image is embedded in the radiation image, and it is possible to easily confirm under what conditions the radiation image was imaged. Generally, the annotation is performed by performing the radiation imaging on a character-shaped structure (annotation marker) made of a material having a high radiation absorption rate so that it appears together with the object. On the other hand, in the second example, the annotation is performed by embedding (superimposing) the object information in the image. In relation to the annotation, the object information according to the second example can include information indicating the posture and the orientation of the object, the right or left of the site, etc. Here, the orientation of the object may correspond to the incident direction of the radiation.

An example of the annotation includes embedding, for example, a character symbol “PA” in the radiation image in a case of imaging of the posture in which the radiation enters from the back, and a character symbol “AP” in the radiation image in a case of imaging of the posture in which the radiation enters the object from the chest, in the frontal imaging of the chest. In another case, the annotation includes embedding, for example, a character symbol “RL” in the radiation image in a case of imaging of the posture in which the radiation enters from the right side of the object, and a character symbol “LR” in the radiation in a case of imaging of the posture in which the radiation enters from the left side of the objection. In addition, the annotation includes embedding a character symbol “R” in the radiation image in a case of imaging of the right hand or the right foot, and a character symbol “L” in the radiation image in a case of imaging of the left hand or the left foot, in a case where a site, which has the left and right sides like the limbs of a human body, is imaged. In addition, the annotation also includes embedding characters such as “standing” in the radiation image in a case where the object has been imaged in a standing state, “sitting” in the radiation image in a case where the object has been imaged in a sitting state, and “lying” in the radiation image in a case where the object has been imaged in a lying state on a bed.

### (Processing Flow)

A series of processing procedures according to the second example will be described with reference to FIG. 8. FIG. 8 is a flowchart showing the processing procedures according to the second example. When the processing procedure of the second example starts, the processing shifts to step S801.

### (Step S801)

In step S801, the optical image obtaining unit 101 controls the camera 40 to obtain an optical image of a region including the object in the radiation imaging. In the second example, the camera 40 is a video camera attached to the radiation generator, and images the object taking the imaging posture on the radiation detector 30 arranged on a recumbent table, and outputs optical images at a predetermined frame rate.

### (Step S802)

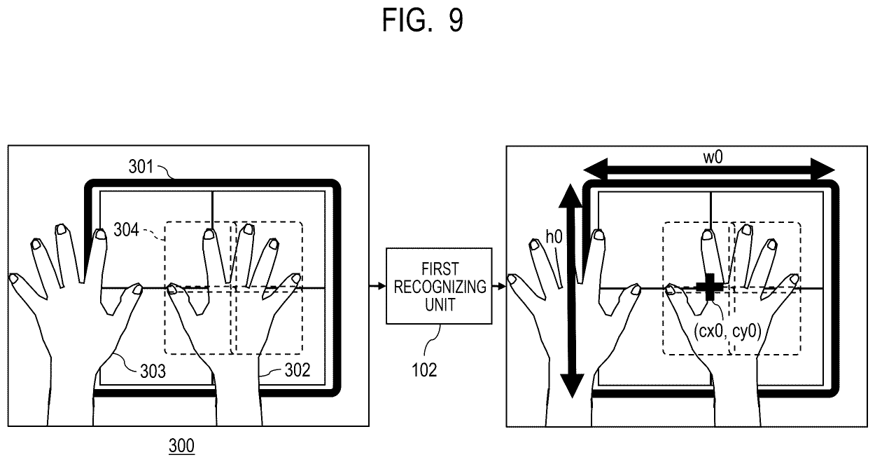

In step S802, the first recognizing unit 102 recognizes a region of interest from the optical image obtained by the optical image obtaining unit 101. Here, the region of interest is a region where an object, which is an imaging-target to be imaged with radiation, is assumed to exist. In the second example, a case where the radiation detector region 301 in the optical image is assumed to be the region of interest will be described. Since the radiation detector 30 is an apparatus for obtaining the radiation image at the time of the radiation imaging and the object exists in the region to be imaged as the radiation image by the radiation detector 30, the radiation detector region 301 in the optical image corresponds to the region of interest where the object exists. Therefore, as shown in FIG. 9, the first recognizing unit 102 recognizes, for example, a circumscribed rectangle of the radiation detector region 301 from the optical image 300, and outputs the center position (cx0, cy0), the width w0, and the height h0 of the circumscribed rectangle as the region of interest, that is information indicating the recognized region of interest.

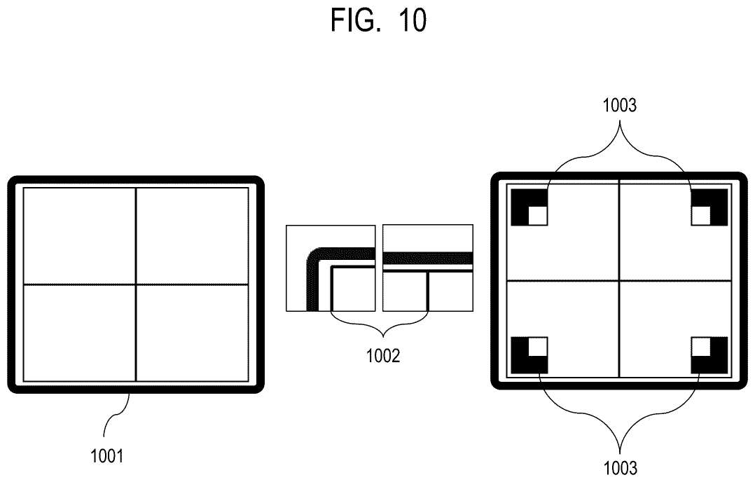

Various methods can be considered for recognizing the radiation detector region 301 in the optical image. For example, there is a method in which the information processing apparatus 100 holds the appearance of the radiation detector 30 as a template to be recognized, and the first recognizing unit 102 extracts and recognizes the radiation detector region 301 from the optical image by the template matching. FIG. 10 shows examples of templates and markers related to the template matching according to the second example. In such template matching, the entire radiation detector 1001 may be used as a template, or characteristic appearance features 1002 such as squares, edges, and logos may be used as a template.

Further, there is a method in which, for example, markers 1003 that is easy to be detected may be pasted on the radiation detector region 301 and the first recognizing unit 102 extracts and recognizes the region of interest based on the marker 1003 in the optical image. For example, the first recognizing unit 102 may recognize a region surrounded by the markers 1003 as the region of interest. In a case where a marker is used, it is possible to design the marker so that the marker can be accurately extracted by combining characteristic amounts extracted by the edge detection and the threshold processing even if the template matching is not performed.

Since three-dimensional deformation such as scaling and rotation occurs on the template or the marker depending on the positional relationship with the camera 40, extraction rules of the template or the marker can be used in consideration of such deformation. Further, for example, a neural network-based inferrer may be generated by a machine learning and used. In this case, the inferrer may be generated by using, training data including, for example, optical images captured at various positions, angles, and lighting conditions including the appearance of the radiation detector or the marker to be used as a template, and a label image in which label is given to the template or marker in the optical image. Instead of the label image, information indicating the position of the template or the marker may be used.

### (Step S803)

In step S803, the second recognizing unit 103 recognizes object candidate information from the optical image 300 obtained by the optical image obtaining unit 101. Since this step is the same process as step S203 in the first example, the description thereof is omitted. If information on the orientation of the object is included in the object candidate information, a label image with a label on the orientation of the object may be used as the training data of an inferrer used by the second recognizing unit 103.

### (Step S804)

In step S804, the recognition result integrating unit 104 determines and outputs object information indicating the object which is the imaging-target of the radiation image by integrating the region of interest output by the first recognizing unit 102 and the object candidate information output by the second recognizing unit 103. In the second example, the object information includes annotation information indicating the posture and the orientation of the object and right/left of site annotated in the radiation image, such as the radiation incidence direction (PA/AP) and the left/right (R/L) of the object.

In this step, as in step S204 of the first example, the recognition result integrating unit 104 determines an object candidate, which is closest to the position of region of interest output by the first recognizing unit 102, from among the object candidates, as the object. The recognition result integrating unit 104 obtains the annotation information, such as the radiation incidence direction (PA/AP) to the object and the left/right (R/L) of the object, based on the object candidate information determined as the object information.

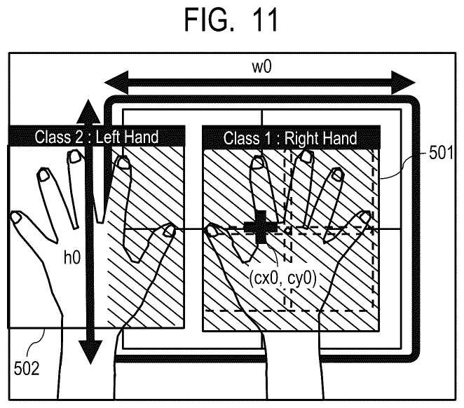

For the object information determination processing, the same technique as in step S204 in the first example may be used, but other methods may be used. As a modified example, a method of narrowing down the object based on the area of the object candidate included in the region of interest will be described. In this method, the recognition result integrating unit 104 determines a region that is the region of interest and the object candidate, compares the areas of the determined regions, and determines the region with the larger area as the object. FIG. 11 shows an example of the region that is the region of interest and the object candidate in this process by shaded regions. This method is based on the assumption that the most of object is located in the region of interest (radiation detector in the second example).

However, in a case where imaging of “fingers” is specified in the imaging order, it may be better to narrow down a region with the smaller area as the object. Therefore, the recognition result integrating unit 104 may set a criterion regarding the size of the region when narrowing down the object based on the information of the imaging-target included in the imaging order.

In the example shown in FIG. 11, it is assumed that the recognition result integrating unit 104 narrows down the right hand, which is larger in the region of interest, from the object candidates which have the same size such as the right hand and the left hand, as the object. As a result, the recognition result integrating unit 104 can output the bounding box 501 and the class number 1 (right hand) which indicates the right or left of the site of the object included in the bounding box 501, as the object information.

### (Step S805)

In Step S805, the operator performs an appropriate operation for the imaging of the radiation image via the operation unit 134, and the radiation image obtaining unit 106 obtains the radiation image. Although omitted in the second example, the consistency judgement may be performed in the same manner as in steps S205 and S206 in the first example, and the display controlling unit 108 may cause the region of interest, the object candidate information, the object information, and the judgement results of the consistency to be displayed on the display unit 135. Also, in the same manner as in the first example, the information processing apparatus 100 may determine whether the imaging of the radiation image is performed or not.

### (Step S806)

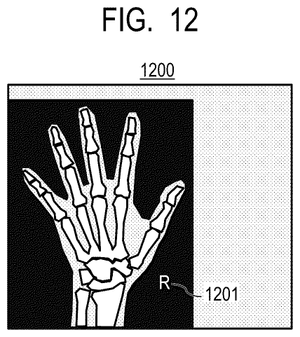

In step S806, the annotation unit 107 annotates the radiation image output by radiation image obtaining unit 106 with the information of object output by recognition result integrating unit 104. For example, in the example shown in FIG. 11, since the class number 1 (right hand) is output as the object information, the annotation unit 107 places a symbol R1201 indicating the right hand in the radiation image and outputs the annotated image 1200 as shown in FIG. 12. FIG. 12 shows an example of output by the annotation unit 107. The output annotated image 1200 may be caused to be displayed on the display unit 135 by display controlling unit 108, stored in the storage 132, or transmitted to the external storage apparatus 60 or the like.

As described above, the information processing apparatus 100 according to the second example further includes the annotation unit 107 which superimposes the object information on the radiation image as the annotation information. In this case, the display controlling unit 108 can cause at least one of the optical image, the region of interest, the candidate information, the object information, the consistency judgement results, and the annotation information to be displayed on the display unit 135.

In this configuration, the information processing apparatus 100 can recognize the object information by the analysis of the optical image and automatically perform the annotation. Further, the information processing apparatus 100 can obtain information for narrowing down the object which is the imaging-target of the radiation imaging from among a plurality of object candidates in the optical image. Thus, the object recognition for the automatic annotation can be performed even if a plurality of object candidates appears in the optical image. Therefore, the information processing apparatus 100 can more appropriately assist the operator's judgment regarding the appropriateness of the imaging position of the object.

Furthermore, the first recognizing unit 102 according to the second example may recognize one of the detector region indicating the radiation detector and the region indicated by the marker as the region of interest by using the optical image. According to such a configuration, the information processing apparatus 100 according to the second example can efficiently obtain the information for narrowing down the object based on the radiation detector or the marker in the optical image, which is the region of interest, even if a plurality of object candidates appears in the optical image.

In the second example, the annotation unit 107 performs annotation to the radiation image. On the other hand, the annotation unit 107 may perform annotation to the optical image or to both of the optical image and the radiation image. Even with such a configuration, the object recognition for the automatic annotation can be performed in a case where a plurality of object candidates appears in the optical image. Therefore, information processing apparatus 100 can more appropriately assist the operator's judgment regarding the appropriateness of the imaging position of the object.

### Third Example

A radiation imaging system, an information processing apparatus, and an information processing method related to a third example of the present disclosure will be described below. Description of the details described in first example will be omitted.

The third example will consider a case where the coordinates (spatial coordinates) of the radiation generator or the radiation detector 30 in a camera coordinate system in which the optical center of the camera 40 is the origin, the optical axis direction of the camera 40 is the Z-axis direction, and the lateral and longitudinal directions of the image are the X-axis direction and the Y-axis direction, respectively, are known. Such coordinates may be obtained by, for example, installing the radiation generator or the radiation detector 30 while measuring the position with respect to the camera 40 using a measure or the like. The coordinates may also be obtained based on the amount of drive from the installation position of the radiation generator or the radiation detector 30 using a configuration in which the amount of drive can be grasped mechanically. Furthermore, the coordinates may be obtained by attaching a gyro mechanism or an acceleration sensor to the radiation generator or the radiation detector 30 to obtain the displacement quantities of the position and the angle from the initial position, and inputting them into the information processing apparatus 100.

In this case, the coordinates (x, y) in the obtained optical image corresponding to the coordinates (X, Y, Z) in the camera coordinate system can be expressed by the following equation 2 when the focal length of the camera is normalized to 1.

$$x = \frac{X}{Z} , y = \frac{Y}{Z} \qquad ( Equation 2 )$$

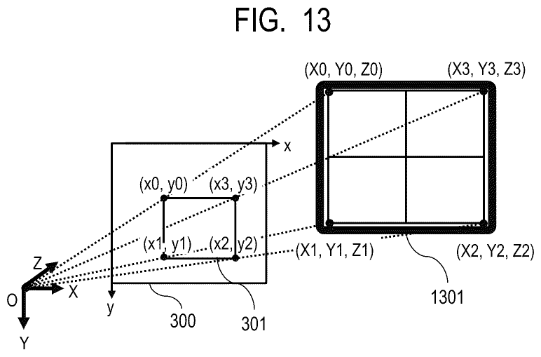

In the third example, with reference to FIG. 13, a method in which the first recognizing unit 102 obtains a region of interest using such coordinates will be described. FIG. 13 is a diagram for explaining an example of output of the first recognizing unit 102 according to the third example. Note that the method for determined the object information from the obtained region of interest and using it for the consistency judgement and the annotation is the same as the method in the first example and the second example, and description thereof will be omitted.

The first recognizing unit 102 recognizes the region of interest from the optical image 300 obtained by the optical image obtaining unit 101. The region of interest is a region where an object, which is an imaging-target to be radiation imaging, is assumed to exist. In the third example, the case where the radiation detector region 301 of the optical image 300 is assumed to be the region of interest, as in the case of the second example, will be described. In the third example, it is assumed that the coordinates (X0, Y0, Z0) to (X3, Y3, Z3) in the camera coordinate system of the four corners of the effective pixel region of the radiation detector 1301 are known. At this time, the first recognizing unit 102 can calculate the coordinates (x0, y0) to (x3, y3) of the four corners of the corresponding effective pixel region on the optical image 300 according to the equation 2. Therefore, the first recognizing unit 102 according to the third example can recognize the region surrounded by the obtained coordinates (x0, y0) to (x3, y3) as the region of interest.

Similarly, if the coordinates of the radiation generator and the coordinates of the collimator at the time of opening and closing are known, the first recognizing unit 102 can calculate the radiation irradiation region by determining the intersection point between the straight line connecting the center of the radiation generator and the collimator and the radiation detector 1301 or the imaging table. In this case, the first recognizing unit 102 can recognize the determined radiation irradiation region as the region of interest. The first recognizing unit 102 may calculate the collimator irradiation region as the radiation irradiation region.

As described above, the first recognizing unit 102 according to the third example determines the radiation detector region where the radiation detector is shown in the obtained optical image by using the spatial coordinates of the camera 40 for imaging the optical image and the radiation detector 30, and recognizes the radiation detector region as the region of interest. The first recognizing unit 102 may also determines the radiation irradiation region where the radiation is irradiated in the optical image by using the spatial coordinates of the camera 40 and the radiation generator, and recognize the radiation irradiation region as the region of interest.

According to the above configuration, even if the region of interest is hidden by the object in the optical image, the information for narrowing down the object, which is the imaging-target of the radiation imaging, can be obtained. Further, the consistency judgement and the automatic annotation can be performed by using the information obtained by such a way.

In the third example, the first recognizing unit 102 calculated the coordinates of the region of interest in the optical image from the spatial coordinates of each component in the camera coordinate system using the equation 2. On the other hand, the first recognizing unit 102 may obtain the coordinates of the region of interest in the optical image from the spatial coordinates of each component in the camera coordinate system by using a table related to the coordinates of the region of interest in the optical image corresponding to the spatial coordinates of each component in the camera coordinate system. In this case, the table may be stored in advance in the storage 132 or the like.

In second and third examples, the optical image obtaining unit 101 and the radiation image obtaining unit 106 obtain the optical image and the radiation image by using the camera 40 and the radiation detector 30 and perform the annotation. On the other hand, the optical image obtaining unit 101 and the radiation image obtaining unit 106 may obtain these images from the external storage apparatus 60 or an imaging apparatus connected to the information processing apparatus 100 via any network. In this case, the optical image and the radiation image may be imaged as long as they were imaged while the object kept the same imaging posture, and the obtaining (imaging) time may be different from each other.

The display screens described in the first example to the third example are examples. Therefore, the arrangement and the aspect of the display of various images and buttons on the display screen displayed on display unit 135 may be set freely.

According to the first example to the third example, the information for narrowing down the object which is imaging-target of radiation imaging can be obtained from a plurality of object candidates in the optical image.

In the above-mentioned inferrers for the recognition processing of the region of interest and the object candidate information, it is conceivable for the magnitude of intensity values, and the order and slope, positions, distribution, and continuity of bright sections and dark sections and the like of an image that is input data to be extracted as a part of feature values and used it for inference processing.

Moreover, the above-mentioned inferrers for the recognition processing of the region of interest and the object candidate information can be provided in the information processing apparatus 100. These inferrers (learned models) may be, for example, constituted by a software module that is executed by a processor such as a CPU, an MPU, a GPU, or an FPGA, or may be constituted by a circuit that serves a specific function such as an ASIC. Further, the inferrers may be provided in another apparatus such as a server connected to the information processing apparatus 100. In this case, the information processing apparatus 100 can use the inferrers by connecting to the server or the like that includes the inferrers through any network such as the Internet. The server that includes the inferrers may be, for example, a cloud server, a FOG server, an edge server, or the like. No that, in a case where a network in a facility, or within premises in which the facility is included, or within an area in which a plurality of facilities are included or the like is configured to enable wireless communication, for example, the reliability of the network may be improved by configuring the network to use radio waves in a dedicated wavelength band allocated to only the facility, the premises, or the area or the like. Further, the network may be constituted by wireless communication that is capable of high speed, large capacity, low delay, and many simultaneous connections.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The processor or circuit may include a central processing unit (CPU), a micro processing unit (MPU), a graphics processing unit (GPU), an application specific integrated circuit (ASIC), or a field programmable gateway (FPGA). The processor or circuit may also include a digital signal processor (DSP), a data flow processor (DFP), or a neural processing unit (NPU).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An information processing apparatus comprising:
a first recognizing unit configured to recognize a region of interest using an optical image including an object, which is obtained in an imaging scene of a radiation image,
a second recognizing unit configured to recognize candidate information indicating a candidate of the object which is an imaging-target of the radiation image using the optical image; and
a determining unit configured to determine object information indicating the object which is the imaging-target using at least one of recognition results by the first recognizing unit and the second recognizing unit.

2. The information processing apparatus according to claim 1, wherein the determining unit is configured to integrate the recognition results by the first recognizing unit and the second recognizing unit, and determine the object information using the integrated recognition result.

3. The information processing apparatus according to claim 1, wherein the determining unit determines the object information using one of a distance and an overlapping area between the recognized region of interest and the candidate indicated by the recognized candidate information.

4. The information processing apparatus according to claim 1, further comprising:
a judging unit configured to judge whether the object information and information included in an imaging order of the radiation image are consistent or not.

5. The information processing apparatus according to claim 4, further comprising:
a display controlling unit configured to cause at least one of the optical image, the region of interest, the candidate information, the object information, and a judgement result by the judging unit to be displayed on a display unit.

6. The information processing apparatus according to claim 4, further comprising:
a display controlling unit configured to cause a warning to be displayed on display unit in a case where the judging unit outputs a judgement result indicating inconsistency.

7. The information processing apparatus according to claim 1, further comprising:
an annotation unit configured to superimpose the object information on the optical image or the radiation image as annotation information.

8. The information processing apparatus according to claim 7, further comprising:
a display controlling unit configured to cause at least one of the optical image, the region of interest, the candidate information, the object information, and the annotation information to be displayed on a display unit.

9. The information processing apparatus according to claim 1, wherein the first recognizing unit is configured to recognize any one of a collimator lamp irradiated region irradiated with a collimator lamp, a detector region indicating a radiation detector, and a region indicated by a marker as the region of interest using the optical image.

10. The information processing apparatus according to claim 1, wherein the first recognizing unit is configured to determine one of a radiation irradiation region to which radiation is irradiated and a detector region indicating a radiation detector in the optical image using spatial coordinates of an optical apparatus arranged to capture the optical image and at least one of a radiation generator and the radiation detector, and recognize the one of the radiation irradiation region and the detector region as the region of interest.

11. The information processing apparatus according to claim 1, wherein the candidate information includes at least one of a position, a kind, and a posture of a human body site.

12. The information processing apparatus according to claim 1, wherein the first recognizing unit is configured to recognize the region of interest related to the obtained optical image using an output from an inferrer which is obtained by inputting the obtained optical image into the inferrer, the inferrer obtained using training data includes an optical image and information indicating a region of interest in an optical image.

13. The information processing apparatus according to claim 1, wherein the second recognizing unit is configured to recognize the candidate information related to the obtained optical image using an output from an inferrer which is obtained by inputting the obtained optical image into the inferrer, the inferrer obtained using a training data including an optical image and candidate information indicating a candidate of an object which is an imaging-target of a radiation image in an optical image.

14. The information processing apparatus according to claim 1, wherein the second recognizing unit is configured to recognize the candidate information in the recognized region of interest.

15. The information processing apparatus according to claim 1, wherein:
the second recognizing unit is configured to recognize a plurality of the candidate information indicating a plurality of the candidates using the optical image; and
the first recognizing unit is configured to recognize the region of interest based on an area in the optical image, which is occupied by each of the plurality of candidates indicated by the plurality of candidate information.

16. The information processing apparatus according to claim 1, wherein:
the second recognizing unit is configured to recognize a plurality of the candidate information indicating a plurality of the candidates using the optical image; and
the first recognizing unit is configured to recognize the region of interest based on distance from each of the plurality of candidates indicated by the plurality of candidate information to the center of the optical image.

17. The information processing apparatus according to claim 1, wherein:
the second recognizing unit is configured to recognize a plurality of the candidate information indicating a plurality of the candidates in time series using optical images obtained at a predetermined frame rate, and recognizes movement of the plurality of candidates in the time series; and
the first recognizing unit is configured to recognize the region of interest based on the movement in the time series.

18. A radiation imaging system, comprising:
an optical apparatus arranged to perform optical imaging of an object;
a radiation generating apparatus and a radiation detector arranged to perform radiation imaging of the object; and
the information processing apparatus according to claim 1.

19. An information processing method comprising:
recognizing a region of interest using an optical image including an object, which is obtained in an imaging scene of a radiation image;
recognizing candidate information indicating a candidate of the object which is an imaging-target of the radiation image using the optical image; and
determining object information indicating the object which is the imaging-target using at least one of recognition results of the region of interest and the candidate information.

20. A non-transitory computer-readable storage medium having stored thereon a program for causing, when executed by a computer, the computer to execute respective steps of the information processing method according to claim 19.
